(19) United States
(12) Patent Application Publication
(10) Pub. No.: US 2023/0175042 A1
(43) Pub. Date: Jun. 8, 2023

(54) A Photonic Method and Apparatus for Detecting Compounds and Pathogens in a Respiratory Sample

(71) Applicants: Jerry Aguren, Spring, TX (US); John Marino, Cypress, TX (US); Asma Mirza, Houston, TX (US)

(72) Inventors: Jerry Aguren, Spring, TX (US); John Marino, Cypress, TX (US); Asma Mirza, Houston, TX (US)

(21) Appl. No.: 17/998,038

(22) PCT Filed: May 6, 2021
(86) PCT No.: PCT/US21/31196

§ 371 Date: Nov. 7, 2022

(51) Int. Cl. C12Q 1/6818; G01N 1/40; B01L 3/00; A61B 5/097; A61B 5/08; G01N 21/64; G01N 33/497

(52) U.S. Cl. CPC C12Q 1/6818; G01N 1/4022; B01L 3/502715; A61B 5/097; A61B 5/082; G01N 21/6428; G01N 33/497; C12Q 1/6825

(57) ABSTRACT

The present invention relates generally to a means of detecting a target molecule, compound or substance through the attractive forces occurring between said target and a target-specific molecular probe whereby said molecular probe expresses a selective high affinity for target and is capable of fluorescent luminescence at a definitive frequency and in a determinable light wave range. Specifically, said molecular probe offers target-specific binding where a resultant duplex molecule fluoresces upon hybridization to a target's unique molecule nucleotide sequence. Said hybridization allows for optical detection of said duplex via a directed, filtered and focused light source which makes said duplex both quantifiable and quantifiable via luminescence (i.e., light wave excitation) and subsequent photo detection utilizing ellipsoidal reflection to amplify detection and measurement improving both sensitivity and specificity.

### CROSS-REFERENCE TO RELATED APPLICATIONS

U.S. Provisional Patent Application No. 63/021,054 filed May 6, 2020 PCT International Patent Application No. PCT/US2021/031196 filed May 6, 2021

### FEDERALLY SPONSORED RESEARCH OR DEVELOPMENT

Non-Applicable

### FIELD OF THE INVENTION

The present invention relates generally to a means of detecting a target molecule, compound or substance through target-specific attraction with a dual-labeled probe whereby said probe expresses a high affinity for a distinct target and is an “excitable”, fluorophore capable of luminescence in a determinable light wave range. Specifically, the oligonucleotide sequence defining the target-specific binding site, takes the form of a dual-labeled probe forming a quenched, stem-loop structure in its native state which fluoresces upon hybridization to a target molecule nucleotide sequence. Said hybridization allows for optical detection of localized probe-conjugate combinations through a directed, filtered and focused light source. In the presence of a target molecule, nano-molecular probe-target conjugates illuminate and are made both identifiable (qualifiable) and quantifiable via luminescence (i.e., light wave excitation) and subsequent photo detection. The presence and quantity of the target molecule may thereby be both detectable and quantifiable through improved optics utilizing ellipsoid reflection enhancing both sensitivity and specificity.

### BACKGROUND

The ability to detect and localize (i.e., qualify), monitor and quantify the expression of specific viral gene expressions in real-time offers unparalleled opportunities for advancements in molecular biology, disease pathophysiology, disease detection and medical diagnostics. Nonetheless, methods currently employed (e.g., selective amplification through multistep Polymerase Chain Reaction sample amplification and “saturation binding” in the case of a microassay such as ELISA) achieve variable rates of sensitivity and specificity with defined shortcomings in their applicability and practicality.

Specifically, in the case of PCR a particular sample must undergo a denaturization (i.e., separation) and synthesis process, with required sequential variations in temperature, for successful completion of each respective step which ultimately results in millions to billions of replicated copies. This process may be repeated on the order of 20 to 40 times (cycles) through the beginning stages of sample procurement, into exponential amplification and sequential thermal cycling resulting in sample isolation and magnification. And while this process is undeniably advantageous in terms of its sensitivity and ability to quantify the amount or number of a specific antigen molecules, the process is also limiting in that (1) the PCR method is multi-step and time-dependent (i.e., non-rapid) thus requiring highly trained personnel and sophisticated and dedicated equipment for sample processing where (2) the target sequence must be first determined and (3) mutations and contaminations are an underlying and ongoing concern.

Currently, in the case of severe acute respiratory syndrome coronavirus 2 (SARS-CoV-2), the identity of the etiological causative agent of coronavirus disease 2019 (COVID-19) is accomplished through a variation of PCR, real-time reverse transcription-polymerase chain reaction (RT-PCR). Through the testing of nasopharyngeal and oropharyngeal fluids, virus shedding is found to be in largest concentrations 5-7 days after symptom onset and continuing in peak concentrations for several weeks after onset. Functionally, swab-derived viral RNA is isolated and converted to complementary DNA (cDNA) which is then amplified (through cycling as above) using Taq DNA polymerase and a series of specific heating and cooling (thermo cycling) events. Manifestly, RT-PCR remains the definitive means of detection, owning to evidenced proven sensitivity, precision and specificity for viral detection over and above available serological tests. But this specificity comes at a cost of (a) time, wherein turnaround time can take from hours to days, and (b) immobility, test being commonly performed in a laboratory setting. Other limitations in RT-PCT include sample storage requirements, sample contamination and, ultimately, cost.

Serological microassays (e.g., ELISA, EIA (enzyme immunoassay) and RT-LAMP (loop-mediated isothermal amplification) in saliva) are a commonly utilized analytical assay for qualitatively and quantitatively measuring and assessing the presence of a target entity within a sample by implementing qualitative detection of IgG or IgM antibodies. Such tests may verify or accompany RT-PCR results, determine a previous immune response against the viral spike (S) protein, include a quantitative aspect and prove helpful to assess acquired immunity against subsequent viral exposure, a basis for assessing reinfection and/or for contact tracing purposes. And while these assays have proven indispensable in modern medical technology, the detection of target molecules is in no way simple. The process entails a multi-step process consisting of pre- and post-analytic steps exclusive of the actual analytic steps of the assay—steps which are numerous and exacting—which rely on a fixed or mobile antigens, enzyme-linked antibodies and enzymatic substrates to produce a subsequent reaction that is in some way detectable or observable. This typically involves several applications with reagents, detergents and a final washing step to remove all non-specific or unbound antibodies which is finally followed by an enzyme facilitated chemical conversion (with a substrate) which results in an absorbance or fluorescence in a specific range or spectrum identifying the adherence of a selective, tagged-antibody to a target molecule or antigen. Though exhibiting a well-documented and lengthy track record, assays are however in need of a laboratory, dedicated equipment and trained personnel, as seen above in PCR testing, but also necessitate binding and washing steps which require exacting, multi-step procedures in order to determine (1) presence or absence of desired target and/or (2) the overall quantify (e.g., concentrations) of said target. This lends more to a retrospective analysis in which antibodies are utilized to better detect the later stages of infection or past infection.

And while antibodies are a logically and naturally derived means of antigen (target) detection, responsible for identification and “tagging” of these antigens typically for a directed, immunological destruction of these targets through a series of actions including neutralization, agglutination, precipitation and complement activation, the antibody detection is only part of the process. To be observable, these antibodies must also be linked to an enzyme and exposed to a substrate specific to the enzyme wherein the resultant reaction (between enzyme and substrate) produces a detectable signal (e.g., a chromogenic change). As is the case with OTC pregnancy tests and rapid Strep tests, this method is particularly helpful to determine the presence or absence of a target but lacks sensitivity when a small number of targets are present. Equally, this method relies upon an appreciable color change for determinations which may indicate the existence of the target without informing the actual quantity of target molecule. What's more, relatively large amounts of substrate must be hydrolyzed by the bound enzyme before any appreciable detection can be achieved.

To overcome this infirmity, enzyme-linked fluorescence assays (ELFA) was introduced to improve sensitivity in samples exhibiting greatly lowered concentrations as compared to those substrates yielding strictly colored products (as above). Whereas ELISA is chromogenic, the substrate used in ELFA is fluorogenic and results in sensitivity rates which are on the order of 100 times more sensitive than ELISA. And, while greater sensitivity rates are achievable, the immunochromatographic process continues to rely on multi-step procedures (including the steps of procuring samples, fixing antibodies, antigen binding, and washing) and therefore requires many steps—both in preparation, actual analysis and reading of results.

And although a multi-step preparation and analysis is untenable in a rapid detection format, the use of fluorescence and chemiluminescence is of particular appeal and importance to inventors. Specifically, although these assay processes are highly labor and equipment intensive and require samples to be collected via blood collection and transported to laboratories in order to determine results, luminescence as a marker or indicator has seen use in the serological detection of disease for a number of years and bears a long track record in this regard. In fact, chemiluminescence or decay from an excited electronic state to a lower energy level resulting in an emitted light, has seen applications in gas analysis for poisons or other impurities, analysis of organic metabolisms, combustion analysis and biomolecule detection. This is especially interesting in the detection of biological markers (e.g., in forensic science) wherein the heme in blood may be utilized to detect even trace amounts of blood. Moreover, fluorescence has the unique ability to both qualify and quantify the amount of a target molecule which is over and above the capability of simple ELISA.

And while serological testing complements “rapid” viral detection, as an indication of current or past exposure, the development of pivotal ‘first-line’ methods of detection, especially in respiratory samples (seeing the highest concentrations during initial stages and peaking in the second week), that are truly “rapid” is an absolute imperative.

It is therefore chiefly critical to rapid detection that inventors have determined a means to provide improved detection methods, evidencing enhanced sensitivity and specificity, through a combination of (1) highly sensitive biological receptivity to a specific target molecule (biomarkers, compounds, pathogens (organic and inorganic), drug substances and poisons alike), whereby target binding produces near-instantaneous duplex formation and subsequent conformational changes resulting in a observable, single-step bio-illumination, in opposite of micro assays, that (2) can be optically detected without sequential (and multi-step) amplification, as in PCR, all via a non-invasive procedure. Moreover, it is equally advantageous for such a (1) biological binding and (2) optical detection unit to require little to no skilled technical intervention or reliance upon a laboratory (other than confirmatory results) which is completely contained within one device that is mechanically simple, largely reusable and capable of being reduced to a hand-held apparatus informed exclusively by utilizing exhaled breath.

### DESCRIPTION OF THE RELATED ART

While immunology (more singularly serology) and fluorescence may find their roots as far back as the 19th century, the combination of the two is only in its nascent stages of development when used for detecting and analyzing respiratory samples for content of specific target substances, molecules and compounds. In fact, breath gas analysis, or “breathprints”, have only seen development starting as late as 1971 when Linus Pauling first demonstrated that human respiration contains a series of gases and compounds that may be utilized to indirectly analyze, monitor and diagnosis patients for any number of biomarkers correlated with diabetes, lung cancer, head and neck cancer, asthma, COPD and cirrhosis. What is more, breath analysis itself has only recently become recognized institutionally as evidenced by the International Association of Breath Research (IABR) only first established in 2005 and having its first breath analysis summit in 2007 (First Breath Analysis Summit/3rd annual meeting of IABR in 2007).

Manifestly, while there have been attempts to detect compounds that are a byproduct or secondary indicator of disease, these endeavors have failed to move beyond secondary analysis and on to a solid correlation to directly observe and analyze causative agents non-invasively and in real time.

Thus, there is a significant, well recognized, and yet unmet need in the art for a means to detect a target molecule, compound or substance through nanomaterial-based technology wherein, for example, a target-specific, oligonucleotide hybridization probe (molecular beacon: MB) acts as an antibody alternative (i.e., an antibody mimetic/probe) which expresses a high affinity for a distinct target molecule, in the present case a viral infective agent. And although the present invention is a viral pathogen, molecular sequestration and visualization can easily be utilized for a compound, biomarker, bacterial pathogen, poison, chemo or biohazard or any other genetic sequence capable of target-specific binding and resultant molecular conformational change. Said MB is particularly useful in the detection and quantification of target molecules where said probe is capable of luminescence after binding, in a specific light wave range, which may be observed and analyzed optically via the methods disclosed herein.

It is the introduction of nucleic acid probes in the late 1990s, by the likes of Tyagi and Kramer, which provides just the alternative to antibody detection, and antibodies themselves, via an oligonucleotide hybridization probe, consisting of short DNA or RNA oligomer sequences, capable of effectively binding to a target molecule and providing a duplex formation with discrete conformational changes. The molecular beacon (MB) itself is a hairpin-shaped structure which is typically 20 to 40 nucleotides in length evidencing a “loop”, “stems” and fluorophore and quencher “end cap” portions. The “loop” structure is roughly 18-30 base pairs which complements the target molecule. Each “stem” portion attaches to the termini of the “loop” structure and are made of short (5 to 7 nucleotides) and reciprocal base pairs ensuring an essentially “closed” conformation. At the 5′ end of the molecular beacon strand is covalently bound a fluorescent dye (i.e., fluorophore or fluorochrome) and at the 3′ end exists a covalently bound quencher dye, which does not fluoresce. In this “closed” conformation, the fluorescence of the sensor is quenched due to the close proximity of the quencher to the fluorophore, ideally completely with very little residual fluorescence, through fluorescence resonance energy transfer (FRET) in the stem-closed formation wherein FRET is used to describe the transfer of energy between the fluorophore and the quencher in a non-radiative process, which is distance dependent. Concisely, the protein-induced conformational change in the molecular beacon resulted in a change of proximity between the fluorescence probes attached to the ends of the probe generating a fluorescence signal change from the “de-quenched” fluorescent dye.

In operation, once the complementary target nucleic acid sequence is “recognized” by the loop structure, the affinity of the loop nucleotides for the reciprocal target nucleotides supersedes the affinity of the base pairs in the stem, due in no small part to the larger ratio of loop structure nucleotides to stem nucleotides, thus forming a duplex with the target molecule. The molecular probe then transitions from an unstable “closed” configuration to the more stable “open” conformation wherein the fluorescent dye (fluorophore) and quencher dye are separated. A process called hybridization.

Therefore, in the absence of a target molecule, the higher stability of bonds between corresponding nucleotides in the stem greatly reduces the chances of a non-specific opening of the MB. Yet, with the introduction of a target molecule, and its recognizable nucleotide sequence, subsequent MB-target binding and hybridization occurs, the combined MB and target form a duplex, said duplex undergoes a conformational change thereby causing the fluorophore to separate from the quencher. The fluorescence signature thereby increases significantly due to diminished energy capture by the quencher caused by increased distance between fluorophore and quencher in the “stem-open” arrangement thus producing observable illumination in the presence of a specific target molecule.

### SUMMARY OF THE INVENTION

The present invention seeks to address, mitigate and/or alleviate the above referenced infirmities and disadvantages of the current state in the art of target molecule detection and to allow for (1) direct observation of a target molecule, (2) through the binding of an illuminable molecular beacon and target compound, that (3) fluoresces, post binding, and is excitable to a specific light range (4) detectable via a specifically emitted wavelength, (5) observable via directed, filtered beams in an optical scanner—all in a (6) reusable, handheld device that gives (7) near-instantaneous results.

In the present invention's broadest terms, the disclosed device allows the rapid detection and measurement of target molecules in an exhaled breath condensate through target-specific “tagging” by a selective probe or beacon, subsequent collection of breath condensate containing said combined duplex of target and “tag” (e.g., probe) and detection and observation of the presence (and quantity) or absence of “tagged” molecules in the collected sample through enhanced light projection to a receiving detector. Succinctly, the present invention is dependent upon both target detection and a conformation-induced fluorescence which is selectively excitable to allow for target-specific recognition (qualification) and quantification through improved optical observation techniques. Such observation utilizes a non-energy dependent reflective, ellipsoid surface to “amplify”, direct and focus fluorescent light toward a sensor/detector for observation, data collection, analysis and conveyance of said data for medical diagnostics.

As well, inventors contemplate similar sensor beacons (e.g., quantum dot (QD) semiconductor particles) to be of particular interest whereby QD photoluminescence has greatly enhanced brightness, in the order of 20 to 100 times the brightness of a fluorescent dye, and high extension coefficient as a potential substitute for conventional donor fluorophores. Further QD size may have implications on coloration and the potential duration of fluorescence which may afford a greater variability between and among QDs as opposed to fluorescent dyes. Yet, although potentially promising, this technology bears some concerns as to safety and toxicity that may potentially adversely affect the test administrator thereby decreasing their utility.

Taking the foregoing into consideration, the term probe (i.e., molecular beacons, quantum dots, antibodies and antibody-like substances including mimetics or other organic or inorganic molecules capable of fluorescence or phosphorescing), in most cases, may be used interchangeably as performing the same basic function of detection and signaling, but as can be seen, each does so in very discrete and disparate ways. It is these differences which informs the present invention in terms of both form and function (i.e., unique operability).

The biological functions of target-probe conjugation and subsequent fluorescence, while integral to the functioning of the present invention provides, though, affords an incomplete analysis of the unique character of the present invention. In addition to the novel use of molecular beacons in non-invasive identification of a target molecule, in this case a specific virus, is further complemented with a new and novel means of biochemical detection. In opposite of a conventional spectrophotometer, where light (illumination) is provided to a sample and the sample emits light in an omnidirectionally and indiscriminately to a detector, the present means of detection directs, focuses and “pinpoints” light toward the detector (via an ellipsoid “concaving” mirror) which efficiently concentrates the detectable light which allows for conservative uses and the ability to detect even very small concentrations of fluorescence.

The present invention itself is thus comprised of (1) a biological component, (2) a collection reservoir and (3) an optical detection device (the later reservoir and device serving a dual purpose). And while each may be viewed independently, it is their relationship to one another, taken as a whole, that provides the combined novelty and utility to the present invention.
A. Biological Components: a target-specific molecular probe (molecular beacon—MB) and target molecule, described herein as a duplex, consist of (a) molecular beacon, (b) a target molecule and (c) a resultant detectable MB-target complex which becomes detectable after undergoing binding, hybridization and a conformational change as to provide sufficient distance between a fluorophore and quencher to allow for luminescence. Further, the amount of target substance may be, in addition to qualification, may be quantified wherein normal ranges are calculated and determined (from absence to presence) and test samples may be extrapolated to evidence a percent of detected substance that is measurable and equated to total body titers (i.e., a control).
B. Collection Reservoir: a hand-held or stationary receiving device (in the form of a receptacle or chamber) consisting of a mouthpiece, a tapered condensate receiving chamber, an exhaust portal and a collection reservoir, capable of both capturing and channeling, with the assistance of gravity, exhaled breath condensate (EBC) into a receiving collection receptacle. The producer of the EBC, the patient, exhales a sufficient quantity of exhalant to coat and saturate an interior chamber of the collection reservoir and cause condensate to exceed the adhesive forces of the receiving chamber and to be collected in an attached reservoir. The collection of condensate may be facilitated through several mechanisms including (1) an induced vortex flow within said chamber better distributing condensate about the interior of the chamber, (2) a hydrophobic surface on the interior of the chamber repelling adhesion and inducing condensate to precipitate down the chamber wall and move in the direction of gravitational flow to the collection receptacle and (3) exogenous application of a refrigerant, either externally applied or internally supplied, before or during exhalant capture.
C. Optical Detection Device (ODD): an instrument (the sensor), which is made to detect a target-specific beacon (once activated through binding) within a collection receptacle of a collected, patient derived respiratory sample wherein the patient's forced breath, exhaled, either contains or does not contain the desired target. Introduced breath, delivered through the present device, thereby allows the target to bind to molecular beacons which may reside on the interior of the chamber of within the collection reservoir. In the case of a MB-target complex, conformational changes induced by MB and target coupling produces bio-illumination which is then emitted directionally (focused), thorough the use of an integrated ellipsoid mirror, to deliver a concentrated luminescence to a receiving detection sensor. In the alternative enzymatically-linked fluorescence induced monoclonal antibody-target duplex, the sensor is capable of detecting an enzyme-antibody-target complex which is illuminable upon excitation with an exogenous light source. This excitation may take the form of an electromagnetic radiation or an absorbed light source dispersing an excitatory wavelength causing said antibody-enzyme-target to emit photons confirming detection. In either case, fluorescence, via fluorescent dye or enzyme linked antibody, can be designed in order to emit specific frequencies of light depending on the designated target, sensor and/or detector requirements, based on a frequency, wherein distinct frequencies or wavelengths may be designated for identifying a particular target within a single sample or several targets within a mixed sample.

And while each one of these above advancements alone constitutes new and novel improvements individually, it is the combination of each of the biological, physical and optical processes and methods together which delivers a truly innovative means of rapid, repeatable and non-invasive target detection.

In a particular preferred embodiments, fluorescence or phosphorescence, can be induced through enzyme-linked bioassay, via conformational changes prompted by target-MB binding and resultant luminescence and or MB with the addition of quantum dots replacing the functional organic fluorophore. It is also within the contemplation of inventors that illumination, fluorescence or phosphorescence may be achieved through one of several means: chemiluminescence, a chemical reaction (the addition of the enzymes to a substrate containing reagent), electrochemiluminescence, radioluminescence (power derived a radiation source), electroluminescence, mechanoluminescence: a mechanical triggering event (ex. as a result of the physical binding of target to probe), piezoluminescence (ex. changing pressures), sonoluminescence (sonic pressure), thermoluminescence, singly, or by a combination thereof.

In addition to the MB-target, fluorescence and sensor device, the present invention apparatus, method of use and system employs one or more ellipsoidal mirrors in the focused collection of detection data. Ellipsoidal mirrors are currently used in various applications like focusing light into optical fiber, focusing lasers, reflective microscopy, spectroscopy, and focusing x-rays and are employed here to exponentially increase detection passively, through reflected light, resulting in an energy independent amplification thereby increasing target sensitivity requiring no additional resources for labeling, illuminating or identifying target compounds. Expressly, this application and use of ellipsoidal reflective properties is utilized to focus and concentrate small amounts of light emitted from a sample, to amplify and increase detection, qualify and quantify the emitted light and thereby allow the present system to identify and measure even minute amounts of target complexes directly. Advantages again exist here with MB-target duplexes over enzyme-linked immunoassay wherein fluorescence is autonomous bioluminescence resulting from conformational changes requiring no exogenous light source for excitation.

Too, the patient interface would consist of a disposable tube containing a sensor, into which the patient would expel breath at a certain rate and volume. Inventors contemplate a means to measure exhalation rates which may be consist of a manual peak flow meter, an auditory indicator (e.g., a whistle) or any automated device, manual or electrical, which determines force and/or length of exhalation.

In operation, if the patient's breath contains the desired target, said target would react with the sensor (i.e., the affinity of the MB, antibody or other antibody mimetic to a target would allow for target binding and hybridization). The tagged-target complex would undergo an illumination event (as described above). The amount of tagged-target present in the volume of breath can be directly “observed”, collected and recorded as related to the amount of light detected. This measure of complex illumination (i.e., measurable light units) may then be used to determine the quantity of the target substance whereby the amount may signify a ‘level’ that allows the observer to determine the presence of target substance, the amount of target substance, or both, in order to determine the effects of the presence and amount of said target substance.

The sensor device may consist of a PCR collection tube or tubes, or similar device(s), or and of a plurality of probes which may be coated with the specific MB or probe designed to bind with the target sample. It is in the further contemplation of inventors to have the interior surface of the collection chamber coated with target-specific probes wherein a specific target, if present, would bind to a probe, and the resulting complex may precipitate into a collection reservoir for detection and analysis. Ideally, the sensor is integrated into a hand-held device, and when incorporated into or inserted into the device, or the device is placed into a collection receptacle having same, the collection is suspended at one focal point of an ellipsoidal mirror.

The patient interface is disposable and may be designed specifically for a single substance or a plurality of substances, sequentially or contemporaneously. The optical detection device can be reused, sensors interchangeable and each can be designed for detecting a variety of substance molecules, light frequencies and wavelengths within the same device. The device may be designed to identify a specific patient interface and calibrate its ability to analyze a patient's breath accordingly (factoring variables, including but not limited to, sensor particle size, target size, complex weight, breath volume and the like).

### BRIEF DESCRIPTION OF THE DRAWINGS

While the novel features and method of use of the application are set forth above, the application itself, as well as a preferred method of use, and advantages thereof, will best be understood by referencing the following detailed description when read in conjunction with the accompanying drawings (in view of the appended claims), wherein:

FIG. 1 depicts molecular beacon (MB), MB-target bound and MB target unbound, in both “closed”, unbound, conformation and “open”, MB-target bound, conformation.

FIG. 2 illustrates the operational key components and functionality of the present device.

FIG. 3 depicts an expanded view of the TE (thermo electric) Cooler.

FIG. 4 shows a block diagram of one embodiment with 2 components (1) a disposable tube and sensor and (2) reusable test instrument.

FIG. 5 is a representational block diagram of another embodiment of a disposable unit and detection device.

FIG. 6a depicts a flow diagram of EBC flow and detection process.

FIG. 6b represents a EBC flow, integral system components and computer analysis.

FIG. 7 evidences a flow diagram of typical target detection and procedures.

FIG. 8 represents a sample and directed fluorescent light via reflective ellipsoid mirror and detection in the present device.

FIG. 9 is an ellipsoidal mirror and the functional components of sample analysis and photon detection.

FIG. 10 depicts a detailed representation of a collection device and detection device utilizing an ellipsoidal mirror.

And while the present invention, integrated system and method of use are amendable to modifications and alternative configurations, embodiments thereof have been shown, by way of example only, in the drawings and are described herein in adequate detail to teach those having skill in the art how to make and practice the same. It should, however, be understood that the above description and preferred embodiments disclosed, are not intended to limit the invention to the particular embodiment disclosed, but on the contrary, the invention disclosure is intended to cover all modifications, alternatives and equivalents falling within the spirit and scope of the invention as defined within the claim's broadest reasonable interpretation that is consistent with the specification.

### DETAILED DESCRIPTION

The following description and accompanying drawings are illustrative and are not to be interpreted as limiting. Various key features are described to provide those having skill in the art the requisite description to made and use the same. However, it is to be understood, that certain features that are routine in the art are not described in detail that would prove pedantic to the skilled artisan. It bears including that the present invention may be practiced, in various cases, in one, some or all embodiments, preferred or simply described, with or without some of all details or features without departing from the spirit and scope of the invention as described.

References to “an embodiment”, “preferred embodiment”, “one embodiment”, “another embodiment” and “yet another embodiment” means that said “embodiments” are inclusive of all other “embodiments” in that a key feature, structure or iteration may be borne across all embodiments where their inclusion is not obviated by physical or structural inabilities to incorporate or combine said features, structures, or iterations or where their incorporation would not render the present invention inoperable for its intended use. To the contrary, “embodiments” as they are described herein are inclusive of all embodiments and not mutually exclusive of other embodiments. It is also in the understanding of inventors that not all features may be included in all embodiments while several features may be included in one embodiment or across embodiments.

Terms used within the present application are to be read as having their ordinary meanings in the art, as defined by the disclosure, and within the context of the surrounding defining language. Those terms, used by inventors, may as well be defined using specific terminology and descriptions are provided in the disclosure enumerating such terms and may have those meanings ascribed to each term as defined by those terms' accompanying language.

Section headings, titles and subtitles may be used strictly for organizational purposes and should not be interpreted as limiting the disclosed subject matter in any way. And while certain descriptions or embodiments may appear under a certain heading, title or subtitle, these descriptions and embodiments pertain to the invention and disclosure on the whole and should be read to apply to the entire description.

The present invention relates to the detection (qualification), analysis and quantification of a specific target molecule within an exhaled volume of breath, through and probe-reporter-detector model and process whereby occurs: (1) a high affinity attachment of said target to a fluorescent-tagged beacon (e.g., probe) to form a beacon-target complex, more technically a duplex, (2) conformational-induced, or enzyme-substrate facilitated fluorescence of said beacon or tag (making said duplex identifiable), (3) collection and/or concentration of said into a collection receptacle, (4) focusing endogenously or exogenously produced fluorescent light toward a detector which senses either the absence or presence of fluorescence and, if present, may use the overall measurable target-probe (via number or light intensity generated to quantify duplex populations through correlations with overall in vivo target concentrations.

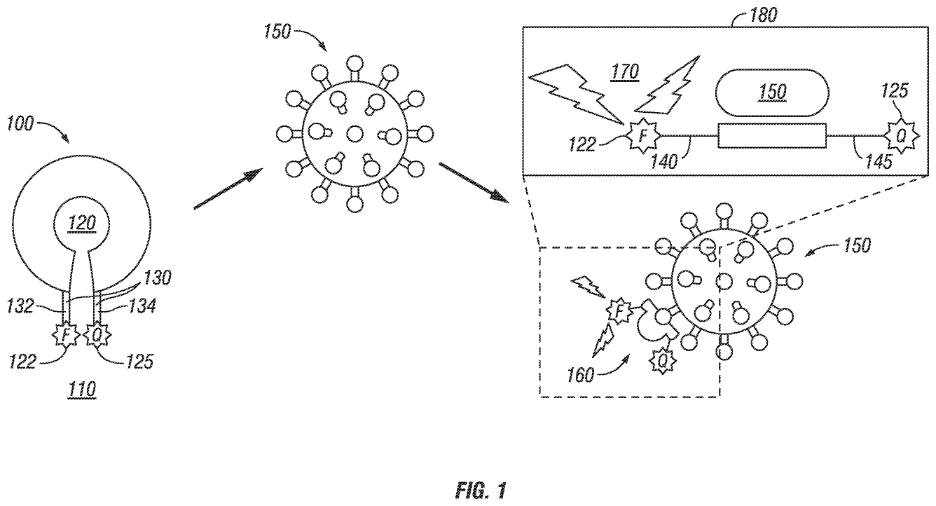

As depicted in FIG. 1, a molecular beacon (MB) 100, commonly described as an oligonucleotide hybridization probe, consists of short chain nucleotides (i.e., oligomeric sequence), having a high binding affinity to a target molecule 150, providing a duplex formation 180 exhibiting resultant conformational changes causing the combination to fluoresce. The molecular beacon 100, as depicted, is a hairpin-shaped structure which is typically 20 to 40 nucleotides in length consisting of a loop 120 and attached stems 130 to which are attached a donator fluorophore (F) 122 and acceptor quencher (Q) 125 existing at the terminus of each stem 130. The loop 120 is approximately 18-30 base pairs which complements (binds to) the target molecule 150. Each stem 130 attaches to the termini of said loop 120 and each are made of short (5 to 7 nucleotides) consisting of reciprocal base pairs (ex. A-T or C-G) ensuring an essentially “closed” conformation 110 wherein, for example, TTTTGGGG may form stem 132 and CCCCAAAA may form stem 134, or vice versa. At the 5′end 140 of the molecular beacon 100 is covalently bound an organic fluorescent dye (F) 122 (i.e., fluorophore or fluorochrome) and at the 3′ end 145 exists a covalently bound quencher dye (Q) 125, which does not fluoresce (although the existence of two organic fluorescent dyes have also been used historically). In this “closed” conformation 110, the fluorescence of the sensor is “quenched” due to the close proximity of the quencher 125 to the fluorophore 122, ideally completely with very little residual fluorescence, through fluorescence resonance energy transfer (FRET) in the stem-closed 110 formation wherein FRET is used to describe the transfer of energy between the fluorophore 122 and the quencher 125 in a non-radiative process, which is ultimately distance dependent. Concisely, the protein-induced conformational change in the molecular beacon 100 results in a change of proximity (increased distance) between the fluorescence dye 122 attached to the end of the molecular beacon 100 probe stem 132 generating a fluorescence signal change (illustrated in duplex formation 180) to a “de-quenched” fluorescent dye 122 effectively converting target 150 (depicted as a viral target) recognition into a fluorescence signal 170.

In operation, once the complementary target 150 nucleic acid sequence is “recognized” by the loop 120, the affinity of the loop 120 nucleotides for the reciprocal target 150 nucleotides supersedes the affinity of the base pairs (A-T and C-G) in the stem 130, due to the number of loop 120 nucleotides in relation to stem 130 nucleotides, thus forming a duplex 180 with the target 150 molecule wherein the molecular beacon (MB) 100 is hybridized to the target 150. The MB-target duplex 180 then transitions from an unstable “closed” configuration 110 to the more stable “open” conformation 160 wherein the fluorescent dye (fluorophore) 122 and quencher 125 are separated due to hybridization.

Therefore, in the absence of a target molecule 150, the higher stability of nucleotide bonds between corresponding A-T and C-G nucleotides in the stem 130 greatly reduces the chances of a non-specific opening of the MB 100. Yet, with the introduction of a target molecule 150, and its recognizable nucleotide sequence, subsequent MB-target binding, duplex 180 formation and hybridization occurs, the combined MB 100 and target molecule 150 form as duplex 180, said duplex 180 undergoes a conformational change thereby causing the fluorophore dye 122 and quencher 125 to separate. The fluorescence signature (fluorescence signal 170) thereby increases significantly due to diminished energy capture by quencher 125 with co-temporal increased distance between fluorophore dye 122 and quencher 125 in the “stem-open” arrangement 160 thus producing observable illumination (via fluorescence signal 170) exclusively in the presence of a specific target 150 molecule.

In the present application, the target (MB 100), here a viral pathogen, is recognized by the probe (MB 100) wherein the target 150 (virus) and the probe 100 bind to form duplex 180. This binding is target-specific and only occurs in the presence of said virus target 150. In the absence of target 150, corresponding A-T and C-G nucleotides in the stem 130 would provide the most stable energy state and would remain in the “closed” conformation 110, providing no probe fluorescence (via fluorescence signal 170).

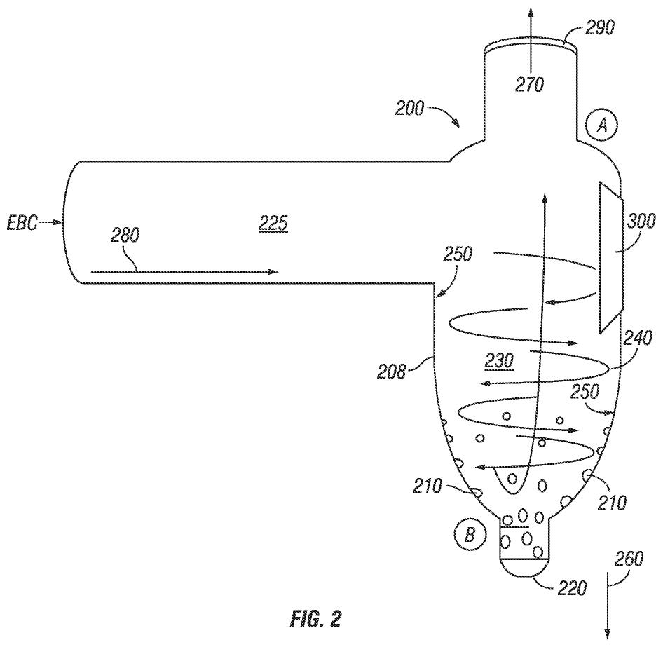

As provided in FIG. 2, duplex 180 formation, as in FIG. 1, may occur either in a collection chamber 200 or in collection reservoir 220. As depicted in FIG. 1, molecular beacon probe 100 would reside in collection reservoir 220, as shown in FIG. 2, whereby condensate 210, provided for by exhaled breath condensate (EBC), enters device 200 through breath tube 225 whereby exhalent enters the device chamber 208 and circles the internal perimeter 230 of device chamber 208 through cyclonic (vortex) action 240 within said chamber 208. It is to be noted that breath tube 225 may be fitted with a one-way valve (not shown) providing a means to supply ECB into the unit without potential reflux. Structurally, internal surface 250 of the device chamber 208 is rounded and tapers as to provide the greatest surface area for EBC (i.e., condensate 210) adhesion. Ideally, internal surface 250 is hydrophobic, repelling condensate 210, and causing condensate 210 to avoid adherence to the internal surface 250. Increased amounts of condensate 210 accumulate on this interior surface 250 and conglomerate (adhere to one another) due to both hydrophobic-induced negative interior surface 250 adhesion and natural cohesion between water molecules (the primary vehicle for EBC). Understanding that the natural form of a water droplet occurs during the “lowest energy state”, the state where the atoms in the molecule are using the least amount of energy, preferable to water, is when a water molecule is surrounded on all sides by other water molecules, creating a “cohesive” sphere or ball. Adding in gravitational pull 260 forces, the individual molecules in the droplet will bind to one another until the droplet (here condensate 210) itself becomes so large wherein the bonds between water molecules (and those bonds to the inner chamber surface 250) is less than natural gravitational pull 260. The droplet then follows gravitational pull 260 to an awaiting collection receptacle 220. And, just as the device breath tube 225, chamber 208 and collection reservoir 220 forms a closed system allowing only a discrete volume of EBC, exhaust valve 270 is incorporated at the superior portion of device 200 to allow for exhalent to escape chamber 275, followed by replacement exhalent 280 supplied so long as the patient continues to exhale EBC. The exhaust filter 270, given the potentially antigenic properties of the exhalant, would exhibit a filter 290, at a point most superior to the present device 200, designated area A, with a sufficient micron pore size as to create a containment exhaust posing no risk to the operating technician or other medical personnel in the immediate area.

The collection reservoir 220, which may take the form of a “PCR tube” or similarly translucent collection reservoir 220, is positioned at the most inferior portion, designated area B, or “trough” area of the present device 200, and opposite from area A and filter 290. It is contemplated that MB 100, or similar probes, may reside on the interior surface of chamber 250, within collection reservoir 220 or a combination thereof.

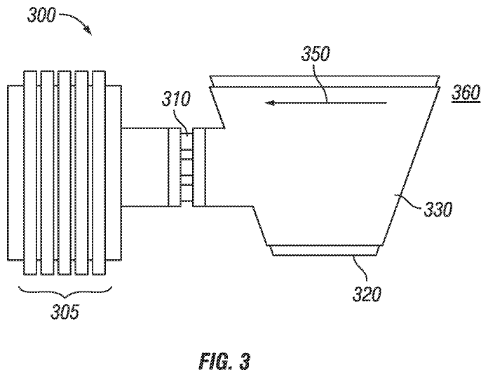

Completing the present device is the externally applied cooling system of FIG. 3. As may be readily understood, externally supplied “cooling” temperature (i.e., those temperatures below ambient air) may hasten the condensation process. In conjunction with a hydrophobic inner chamber surface 250, exogenous cooling facilitates an accelerated condensation and thus a more “rapid” detection device. The exogenously supplied coolant can be supplied through (1) pre-freezing of collection chamber 208, (2) cold application to the external surface of collection chamber 208, (3) or via a dual luminal collection chamber 208 (not shown) whereby a cooling agent may be supplied between an internal lumen surrounded by an exterior capsule (allowing for cooling of the space between the internal lumen and outer capsule. Coolant may supply through pre-refrigeration/pre-freezing, ice, thermoelectric cooling, vapor compression cooling, refrigerant gels, cooling liquid, “cold pack” cooling (i.e., those coolants using water and ammonium nitrate, calcium ammonium nitrate or urea causing an endothermic reaction) and/or phase change gels, singly or a in combination. As depicted, the cooling apparatus 300 of FIG. 2, is not to scale and is merely representational of a cooling apparatus as illuminated further in FIG. 3.

FIG. 3 depicts a thermoelectric external TE cooling apparatus 300 whereby Radiator 305, dissipates heat removed from cyclonic section 330 (corresponding to collection chamber 208 of FIG. 2) wherein said external Thermal Electric cooler 300 is attached to said cyclonic section 330 of breath tube 225 wherein vortex condensation unit 320 is facilitated in its action by heat collection, heat conduction 350 and/or cooling beyond ambient air 360, conduction and cyclonic section 330 which moves heat from cyclonic section 300 (e.g. collection chamber 208) and into TE Cooler 310.

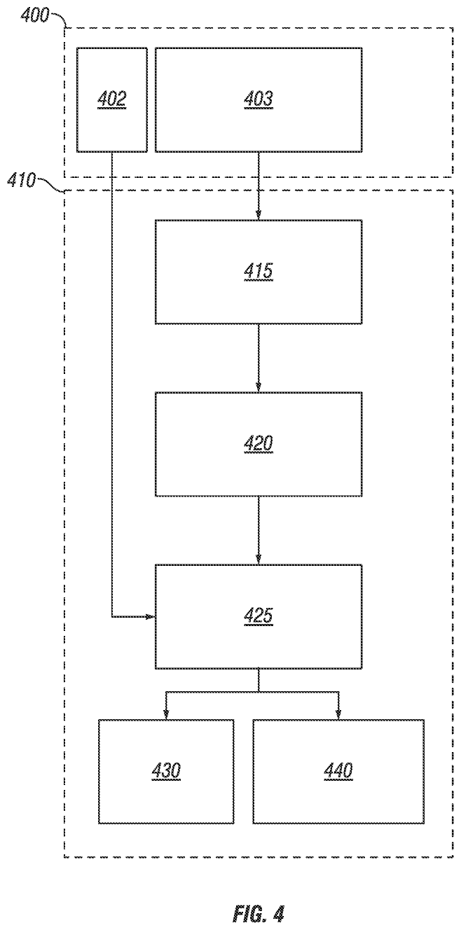

FIG. 4 depicts a block diagram of the present invention, testing system and method of use. The system can be divided into two main components shown by 400 (containing photo tag 402 and disposable tube and sensor 403) and 410, which contain the testing device instrumentation, respectively. The disposable tube system has an additional data tag 102. The data tag 402 is an electronic integrated circuit, which may be integrated or insertable, that contains identifying information about the target-specific test, manufacturer, lot number, expiration and other defining data as may be desirable to include (and may be a QR code). Also, within the disposable breath tube 403 (i.e., disposable tube and sensor 403) for the actual detection and collection of target substances or compounds. The testing device/instrument 410 contains several subsystems, including photo collection 415, photo detection and application 420, signal processing 425, display 430, and communication 440 to an exterior computer for receipt of collected data, monitoring, analysis, presentation and storage of tests results.

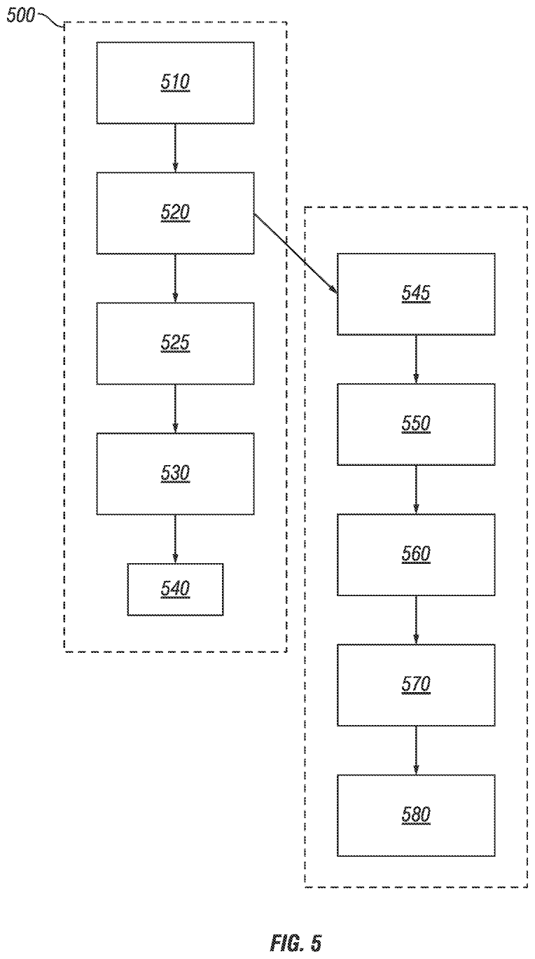

One preferred embodiment, as shown in FIG. 5, is of the disposable breath tube 225 represented as disposable breath tube system 500 of present device 200 containing a breath tube unit 510, a sensor unit 520, a spirometer unit 525, a target (virus) filter unit 530 and a data tag 540 (which may be either a barcode or QR code). The disposable breath tube system 500 and test system 505 are diagrammatically adjoined and may be reversibly connectable as to allow for disposal of subunit 500 and retention of subunit 505. Subunit 500 is defined by its components, indicated by disposable breath tube system 500 of present device 200 containing a breath tube unit 510, a sensor unit 520, a spirometer unit 525, a target (virus) filter unit 530 and a data tag 540, wherein sensor 520 and data tag 540 may be integrated, replaceable or removable. Moreover, spirometer 525 and virus filter 530 may be integrated or replaceable depending on variable perimeters (e.g., anticipated expiration rate due to health and age, virus particle size, and the like). The test system and present invention 505 and its individual components are defined and indicated by photon collection device 545, photodetector 550, amplifier 560, analog to digital converter 570, and microprocessor 580. While inventors have depicted two subunits, 500 and 505, designed for reversible attachment and detachment, it is within contemplation of the inventors that the two subunits, 500 and 505, may exist as a single unit.

In operation, the device user places a rubber tube, mouthpiece, cover or like apparatus (not shown) at the opening of the 225 or like breath collection device, in patient's mouth and exhales, via a self-actuated air movement either normally or forcibly. The user's exhalation (ECB) collects on the collection chamber interior 250, accumulates, aggregates (i.e., precipitates) and “pools” into the collection reservoir 220—serving as a target sensor 520. The patient's exhalant continues in a vortex/cyclonic collector (see FIG. 2 and FIG. 3), creating condensate 210 ultimately collecting in the collection reservoir 220 (serving as sensor 520). ECB, unencumbered by the weight of condensate 210, thus follows the path of least resistance up through chamber 208, through exhaust valve 270, followed by replacement exhalent 280, traversing upper filter 290 and finally existing device 200 through escape chamber 275 wherein patient's expelled air is ultimately exhausted from the disposable tube system 200.

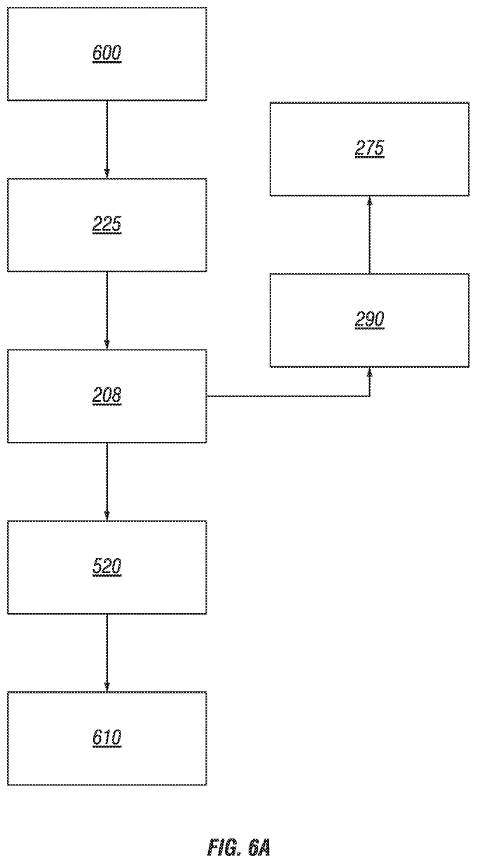

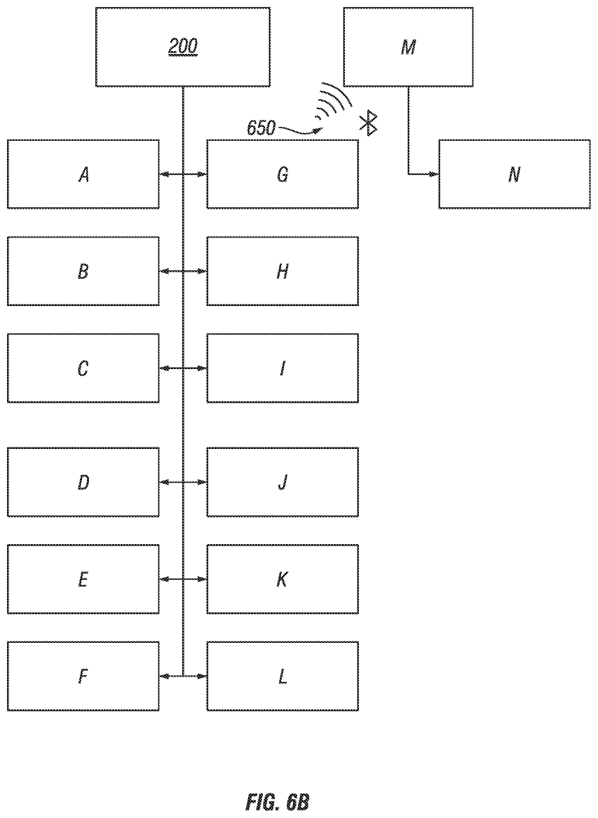

In one preferred method, the sensor 520 contain MBs 100 that are specific for a particular target 150 (e.g., bacteria, virus, biomarker or inorganic marker). If the invention's user's breath (EBC) contains the desired target (i.e., pathogens or other sought particles or compounds) specific to the molecular beacon 100, the target 150 will bind to the molecular beacon 100 and form the duplex described above. The photon emission (as seen in fluorescence signal 170 of FIG. 1) may be the result of a chemical reaction, electrical energy (i.e., via light wave introduction), conformational changes or the like. Light, either endogenously produced or exogenously excited then collected by a photon collector 545, in subunit 505 of FIG. 3. The photon collector 545 focuses photon light emitted from the sensor 520 onto a photodetector 550, where the detected photons may be converted to an electrical current. An electronic amplifier 560 amplifies the signal (electronic current) where the signal is then converted to a digital signal resulting in a digital indication of test results by an analog-to-digital converter 570. The converted digital signal and resultant number is then transmitted to and sampled by a microprocessor 580 for data analysis (see specifically FIG. 6b).

FIG. 6a illustrates the path of the ECB. As shown breath tube 225 is fitted with mouthpiece 600 wherein said mouthpiece 600 is sterilized prior to shipment and is made of a hypoallergenic material. Further, depending on application, breath tube 225 may as well be sterilized prior to use. It is also acknowledged by inventors that a one-way valve (allowing breath into device 200 but not retrograde out of device 200) which may be placed within mouthpiece 600, breath tube 225, or at a defined by the junction between said mouthpiece 600 and said breath tube 225. The ECB (exhaled breath condensate) then exits breath tube 225 and enters collection chamber 208 wherein the ECB allows for circulation of air via a vortex or cyclone-type action 240. Condensate 210 then accumulates via ‘vortex condensation’ within collection chamber 208. Exhaled air is then allowed to escape through exhaust filter 290 wherein said exhaust filter 290 filters exiting air or expended exhaled breath condensate (eEBC) from viral, bacterial and other small particles) but does not significantly inhibit air flow. Exhaust filter 290 may be any micron size sufficient to ensure the capture of harmful exhalant. Exhalant then exits the through escape chamber 275. Condensate 210 then accumulates in collection reservoir 220 whereby said collection reservoir 220 serves as sensor 520 for EBC condensate 210 analysis via the Multi-pixel Photon Counter (MPPC/SiPM 6 mm×6 mm) Sub Assembly 610.

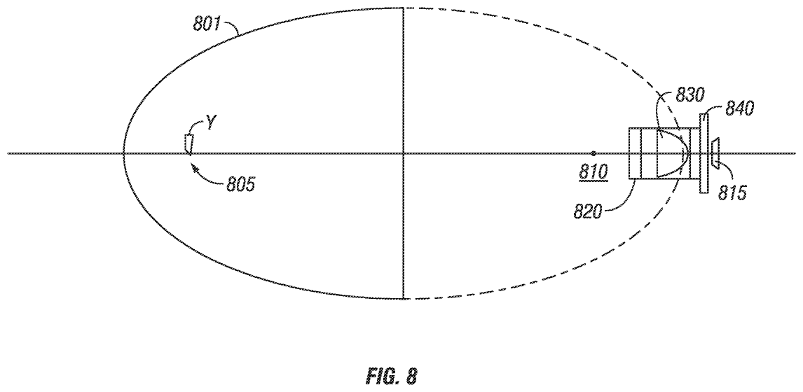

FIG. 6b provides a block diagram of the present device 200 and system architecture. As can be seen in the descending left column: Box A is the device's collection chamber (208) and primary enclosure, Box B signifies the thermoelectric (TE) Cooler Sub Assembly (illustrated further in FIG. 3) for accelerating condensation of condensate 210 (which could also be pre-refrigeration/pre-freezing, ice, thermoelectric (TE) cooling, vapor compression cooling, refrigerant gels, cooling liquid, “cold pack” cooling (i.e., those coolants using water and ammonium nitrate, calcium ammonium nitrate or urea causing an endothermic reaction) and/or phase change gels), Box C the mounting structure into what the device is placed for analysis, Box D is the Ellipsoid Mirror (see also FIGS. 8-10 and ellipsoid mirror 801), Box E is the Multi-pixel Photon Counter (MPPC/SiPM 6 mm×6 mm) Sub Assembly 610 used as the photosensitive mechanism in the present device 200 (currently manufactured by Hamamatsu) and Box F represents the devices Firmware. As is evident in the descending right column: Box G is signifies the Sample Tube Volume (ex. 0.15 ml PCR tube BrandTech® wherein 0.33% is filled plus or minus 1 mm), also signified as a collection tube 220, which may be included with the device 200 as sterilized breath tubes or supplied separately (and handled by laboratory technicians trained in handling biological substances), Box H is the operator controlled or operator informing a Display or displays for providing a graphical representation indicating test readiness, test progress and test completion, Box I is the Micro Controller Board for acquiring sensor information and guiding and informing the test operator and then uploading exam results to an externally residing computer (Box M) via WiFi® or Bluetooth® 650 wherein said computer (Box M) may have programmed into its software an application specific for the present devices' operation (see Box N), Box J is the Sensor Board, Box K is the High-Definition Camera for scanning a 5 mm QR Code (or bar code) at 8 inches, Box L is the operable Battery and Charger controlling operable equipment, ideally for 8 hours, in Boxes D, E, F, H, I, J and K wherein said Battery or Batteries may be chemical, lithium expendable or rechargeable and, where rechargeable, said Charger supports domestic and international power.

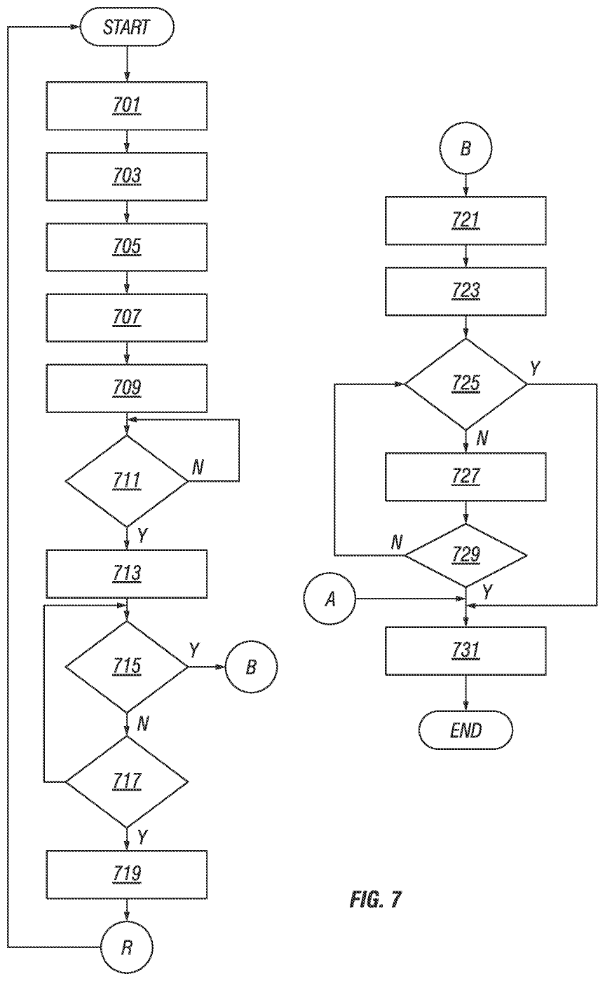

As illustrated in FIG. 7 a flow chart is provided for conducting a typical target detection. By way of example, from Start, a QR code is scanned from the breath tube label (step 701) to verify that the breath tube is compatible with the device and is coded for the patient, a new breath tube is inserted into the present device (invention) (step 703), the device then indicates when calibration is completed (step 705). The breath tube is either placed in patient's mouth or the patient is guided to the breath tube (707) wherein, once the patient is fixed in an upright position (step 709) wherein the device remains +/−20° from the vertical position and patient is coached to remain in an upright position. The patient is then encouraged to form a tight seal with patient's mouth around mouthpiece and to exhale, either normally or forcefully, dependent upon the parameters of the test, and the test is initiated or “triggered (step 711). If the technician fails to “trigger” the device, operations are returned to the previous step (step 709). If the “trigger” (step 711) is successful, the process moves to the next step (step 713) whereby the technician either places the complete collection device into a detector or sample is simply removed from the inferior portion of the device and placed into a detector and technician then monitors sample volume, patient status, initiates adsorption LEDs and samples MPPC output where appropriate. Once sample volume is met (step 715), the process moves to (B). If the sample volume is not met or the test times out (step 717) the test may continue until step 715 (sample volume met) is successful. Yet, if the sample has timed out (due to a patient's inability to produce adequate sample or to complete the test within a set time) the test itself has failed (step 719) and must be repeated (R) which returns to Start. Step 715 is also the determinative step (as depicted in FIG. 1) wherein those probes specific to a single antigen (or other molecular substance desired) would undergo bonding and hybridization in the presence of said antigen. Alternatively, if the sample volume is met (step 715) the collection of sample is complete (step 721) and time of incubation, if any, begins (step 723). This step 723 may be either the time period in which the process takes to complete or the time period for confirmatory results. Once the incubation period times ends (step 725), the sample is tested via MPPC (step 727) and the determination is made as to whether a target is absent or target is present (wherein a pre-set quantification (e.g., determination of a viral load) may be conducted) (step 729). If the target (e.g., virus) is present, but quantification has not occurred (step 729), then incubation time may be extended returning to step 725. If the incubation time in step 725 yields a negative result, those results are analyzed and recorded where data is transferred and uploaded into a computer (upload MPPC output, upload breath test state, upload QR label data in step 731) or if step 725 continues to determine presence of a target (i.e., virus) in steps 727 and 729, those data are then transferred and uploaded to a computer the process is complete (END).

As depicted in FIG. 8 sample Y tube is positioned within the ellipsoid mirror 801 with a focus point 805 (focus point 1) is imaged to the MPPC 815 located to the right of the ellipsoid mirror focus (focus point 2) 810 wherein mirror 801 is utilized to direct the fluorescent light through aperture 820, through condenser lens 830 to optical bandpass filter 840 to MPPC 815 wherein photons are counted up to and including ingle photon detection with high photon detection efficiency rates that expresses high resistance to excess light and limited to no distortion due to magnetic fields, allowing for not only the detection of the presence of fluorescent light but also each occurrence.

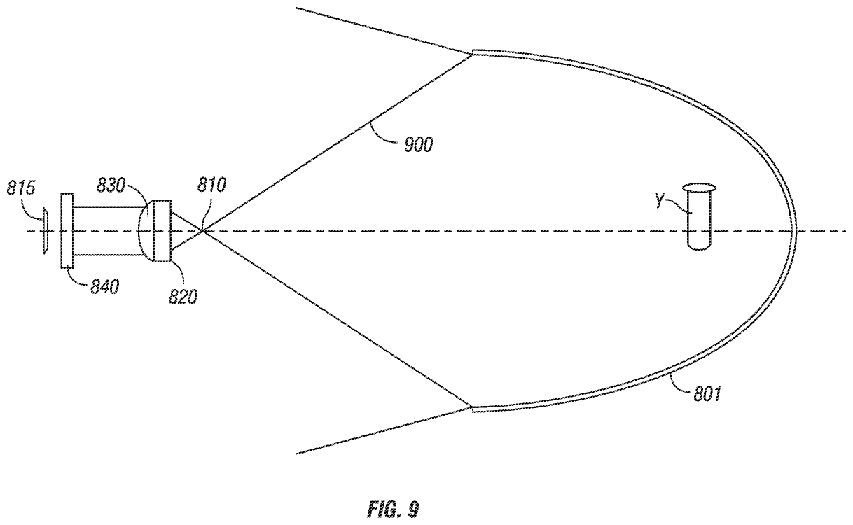

Equally in FIG. 9, the optical representation of the focus 900 of sample Y is fixed at focus point 810 wherein ellipsoid mirror 801 is used to direct the fluorescent light of sample Y through aperture 820, through condenser lens 830 to optical bandpass filter 840 and to Multi-Pixel Photon Counters (i.e., photomultiplier) 815 wherein the fluorescent light is conserved and concentrated for transmittance and detection by the MPPC.

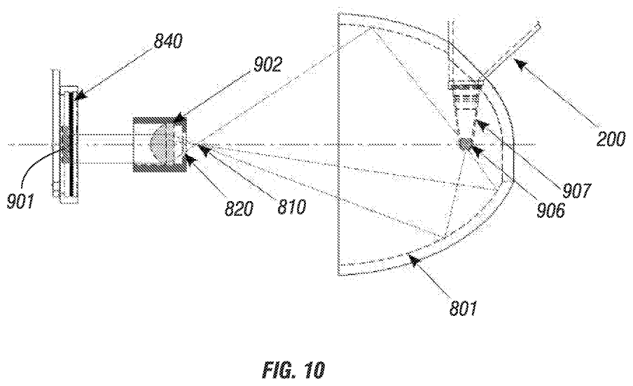

FIG. 10 shows in greater detail the optical bandpass filter 840 of FIGS. 8 and 9 whereby light focused at point 810 is transmitted through aperture 820, through collimation lens 902 and onto photo detector array 901. Light occurring at the biofilter 906 resides at the bottom of PCR tube 907 for exhaled breath condensate, represented above as the collection reservoir 220 of FIG. 2, which is then reflected across the surface of ellipsoid mirror 801 to focus point 810 supplying non-energy consuming, reflective amplification.

From the above disclosure, many alterations and modifications, including preferred embodiments, of the present invention to a person having requisite skill in the art. Therefore, descriptions should be interpreted as merely illustrative and exemplary as providing the best mode or modes contemplated by inventors instructing the skilled artisan to make and use same which is susceptible to broad utility and application. Expressly features, structures, sizes, variations and configurations may be amended, restructured and reconfigured without departing from the scope and spirit of the present application.

The foregoing disclosure is not to be viewed in a limiting sense or to otherwise exclude any embodiments, adaptations or equivalent arrangements naturally following from disclosure of the present application. In opposite, the claims of present invention should be read giving the broadest interpretation to the inventive aspects of the invention described herein and only limited by the claims appended hereto including equivalents thereof.

We claim:

1. An apparatus for rapidly detecting a target molecule, compound or substance via a molecular probe whereby said apparatus comprises the following:
a breath tube for the conveyance of condensate of EBC (exhaled breath condensate);
a collection receptacle;
said collection receptacle being circular as to allow for a vortex flow within said collection receptacle;
a collection reservoir;
said collection reservoir residing at a point inferior to said collection receptacle;
said collection receptacle capable of accumulating condensate from EBC;
a condensate accelerator; and
said condensate accelerator applied to or with said collection receptacle;
an exhaust portal;
said exhaust portal residing at a point superior to said collection receptacle;
said exhaust portal providing for the exit and expulsion of expended EBC (eEBC).

2. The apparatus of claim 1 wherein said collection receptacle has an inner surface that is hydro-phobic as to facilitate condensate movement down said surface as influenced by gravitational forces.

3. The apparatus of claim 1 wherein said condensation accelerator is device which is endogenous to or exogenously adjacent to said collection receptacle.

4. The apparatus of claim 3 wherein said condensation accelerator is provided by a pre-refrigeration/pre-freezing prior to administration of ECB.

5. The apparatus of claim 3 wherein said condensation accelerator may comprise one or more of the following:
ice;
thermoelectric (TE) cooling;
vapor compression cooling;
refrigerant gels;
cooling liquid;
“cold pack” cooling (i.e., those coolants using water and ammonium nitrate, calcium ammonium nitrate or urea causing an endothermic reaction) and/or phase change gels;
heat sinks;
heat conductors;
exothermic reactants; and/or
endothermic reactants.

6. The apparatus of claim 1 wherein said collection receptacle, collection reservoir or a combination thereof contains molecular beacons (MBs).

7. The apparatus of claim 6 wherein said molecular beacons contain a nucleotide sequence comprising a loop and two stems whereby said stems express two fluorescent dyes or a fluorescent dye and a quencher wherein said stems express reciprocal affinity, through corresponding base pairs in a closed conformation.

8. The apparatus of claim 7 wherein said molecular beacons' loop expresses a target-specific affinity for a corresponding nucleotide sequence on a target molecule, compound or substance and wherein said target, if present, binds to said MB.

9. The apparatus of claim 8 wherein said binding of said MB and said target causes a conformational change in said MB and subsequent hybridization, creatin g a MB-target duplex, whereby said fluorescent dye and said quencher are distanced from one another causing said fluorescent dye to fluoresce.

10. The apparatus of claim 9 wherein said fluorescence allows an observer to detect the presence of a target molecule, compound or substance as a result of MB-target binding, MB hybridization, MB conformational change, fluorescent dye and quencher created distance and a non-quenched, fluorescent dye which is made to fluoresce.

11. The apparatus of claim 10 whereby said MB-target fluorescence duplex may be observable through a Multi-pixel Photon Counter (MPPC).

12. The apparatus of claim 11 whereby said MPPC is aided through the use of an ellipsoid reflective mirror which acts to direct and focus fluorescent light to said MPPC.

13. The apparatus of claim 12 whereby ellipsoid reflected light may be amplified through a non energy consuming process facilitating light passage through an aperture, through a condenser lens and through an optical bandpass filter as to concentrate said reflected fluorescent light directly into said MPPC.

14. The apparatus of claim 13 wherein said mouthpiece may exhibit a one-way valve, said exhaust portal may exhibit a filter, or a combination thereof.

15. A system for rapidly detecting a target molecule, compound or substance via a molecular probe, collection device and optical detection device whereby said system comprises the following:
a target-specific molecular probe and target molecule, creating a duplex, consist of (a) molecular beacon, (b) a target molecule and (c) a resultant detectable MB-target complex which becomes detectable after undergoing binding, hybridization and a conformational change as to provide sufficient distance between a fluorophore and quencher to allow for luminescence;
an exhaled breath condensate (EBC) receiving device comprising a mouthpiece, a tapered condensate receiving chamber, an exhaust portal and a collection reservoir, capable of capturing exhaled breath condensate (EBC) into a receiving collection receptacle to produce a sample; and
an optical detection device which focuses emitted fluorescent light thorough the use of an integrated ellipsoid mirror, to deliver a concentrated luminescence to a receiving detection sensor.

16. The system of claim 16 wherein said receiving tapered condensate receiving chamber must be maintained in a largely upright position and may employ means of accelerating condensate capture using said collection reservoir via one or more of the following, singly or in combination, the following:
vortex flow;
cyclonic flow;
a hydrophobic interior;
gravitation force; and/or
applied cooling.

17. The system of claim 17 wherein said EBC receiving device may be (1) integrated into an optical detection device or (2) may be placed into a secondary device as to assure proper placement of said sample.

18. The system of claim 18 wherein said exhaled breath condensate (EBC) receiving device, said optical detection device, or a combination thereof, may be tagged, labeled or otherwise inventories as to provide for proper target molecule, compound or substance identification.

19. The system of claim 15 wherein said exhaled breath condensate (EBC) receiving device and/or optical detection device, or a combination thereof, may be connected to a display device, microcontroller, a computer either directly or through a wireless means (e.g., WiFi or Bluetooth).

20. A method for rapidly detecting a target molecule, compound or substance via a molecular probe, collection device and optical detection device whereby said method comprises the following steps:
a. scanning a QR or bar code on device, patient, or a combination thereof, to ensure accuracy;
b. conducting a rapid detection test by the following:
i. zeroing said collection device;
ii. checking or inserting new or charged batteries into collection device;
iii. inserting a new breath tube into collection device;
iv. ensuring both patient and device are in the upright position;
v. ensuring patient forms a tight seal around mouthpiece;
vi. triggering device;
vii. instructing patient to exhale into the collection device;
viii. collecting sufficient condensate and sample volume; and
ix. instructing patient to cease exhalation;
c. placing collection device into a detection device, or if detection device is integrated, conducting a scan of the sample using focused light from the reflective ellipsoid mirror to concentrate fluorescent light, if present;
d. alternatively, the collected sample may removed from the collection device and placed into a detection device; and
e. conducting a sample reading using ellipsoid mirror to direct, concentrate and focus light through an aperture, through a condenser lens and optical bandpass filter and into a Multi-pixel Photon Counter whereby a sample can be analyzed quantitatively and qualitatively to determine presence and amount of target molecule, compound or substance;
f. determining presence and quantity of target molecule, compound or sub stance;
g. causing transmittance or affirming automated transmittance of collected data to a computer to an external computer via wireless, Wi-Fi or Bluetooth transmission; and
h. accessing collected, analyzed, stored, displayed or other computer or application processed data for qualitative and quantitative results.
